(19) United States
(12) Patent Application Publication
(10) Pub. No.: US 2017/0348453 A1
(43) Pub. Date: Dec. 7, 2017

(54) Odor Mitigating Media Caddy

(71) Applicant: Jeffrey Halsey, Davenport, IA (US)

(72) Inventor: Jeffrey Halsey, Davenport, IA (US)

(21) Appl. No.: 15/615,200

(22) Filed: Jun. 6, 2017

Related U.S. Application Data
(60) Provisional application No. 62/346,285, filed on Jun. 6, 2016.

(51) Int. Cl. A61L 9/12; A47G 25/60

(52) U.S. Cl. CPC A61L 9/12; A47G 25/60; A61L 2209/14

(57) ABSTRACT

An odor mitigating caddy includes first and second frame members each having opposed front and rear edges, respective rear edges being pivotally coupled together with a living hinge such that the frame members are selectively movable between a closed configuration at which the front edges, respectively, are generally adjacent one another and an open configuration at which the front edges, respectively, are separated from one another. The first and second frame members are defined by peripheral edges and include first and second grate members extending between respective peripheral edges, respectively. A porous odor mitigating sheet is held between the frame and grate members. First and second frame members include aligned first and second hook members so that the caddy is selectively suspended from a closet rod or the like.

### REFERENCE TO RELATED APPLICATIONS

This application claims the benefit of provisional patent application U.S. Ser. No. 62/346,285 filed Jun. 6, 2016 titled Media Deployment Device which is incorporated in its entirety herein by reference.

### BACKGROUND OF THE INVENTION

This invention relates generally to air freshening devices and, more particularly, to a caddy for selectively retaining and protecting an odor reducing medium such as a dryer sheet.

Mitigating or eliminating stale, musty, or debris related odors from closets, utility rooms, trunks, or automobile interior areas is a common goal of various products. For instance, air freshener sprays, odor eating solids, and hanging fresheners are well known. In addition, odor absorbing or scented dryer sheets (typically tossed into a load of laundry being dried in a residential dryer) are placed by residents on closet shelves, automobile floorboards, car trunks, etc. to freshen those areas. Dryer sheets have even been found to have rodent and insect repellant properties. Although dryer sheets are indeed effective for their intended purposes, dryer sheets have the inevitable disadvantages of getting pushed to the side, becoming buried under boxes, blowing away, or otherwise not staying where they are intended and needed.

Therefore, it would be desirable to have a framework (caddy) for holding, retaining, and protecting an odor mitigating or eliminating sheet, such as a dryer sheet, that enables many alternative uses of the sheet to be accomplished much more successfully than using the sheet by itself. Further, it would be desirable to have a caddy that retains a dryer sheet or similar media for placement in locations such as attics, basements, garden sheds, outdoor storage units, RV's, boats, and the like in a manner that it will stay where placed or hung.

### SUMMARY OF THE INVENTION

An odor mitigating caddy according to the present invention includes first and second frame members each having opposed front and rear edges, respective rear edges being pivotally coupled together with a living hinge such that the frame members are selectively movable between a closed configuration at which the front edges, respectively, are generally adjacent one another and an open configuration at which the front edges, respectively, are separated from one another. The first and second frame members are defined by peripheral edges and include first and second grate members extending between respective peripheral edges, respectively. A porous odor mitigating sheet is held between the frame and grate members. First and second frame members include aligned first and second hook members so that the caddy is selectively suspended from a closet rod or the like.

Therefore, a general object of this invention is to provide an odor mitigating caddy suitable for holding, protecting, and preventing loss of an odor mitigating sheet or a fragrance emitting sheet.

Another object of this invention is to provide an odor mitigating caddy, as aforesaid, having a pair of frames that sandwich a dryer sheet and have a hook member making the caddy able to be hung where needed.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of an odor mitigating caddy according to a preferred embodiment of the present invention, illustrated in a substantially closed configuration;

FIG. 2 is a top view of the odor mitigating caddy as in FIG. 1;

FIG. 3 is a side view of the odor mitigating caddy as in FIG. 1;

FIG. 4a is a perspective view of the odor mitigating caddy as in FIG. 1, illustrated in a partially open configuration;

FIG. 4b is a perspective view of the odor mitigating caddy as in FIG. 1, illustrated in a closed configuration.

FIG. 4c is a perspective view of the odor mitigating caddy as in FIG. 1, illustrated in a fully open configuration.

### DESCRIPTION OF THE PREFERRED EMBODIMENT

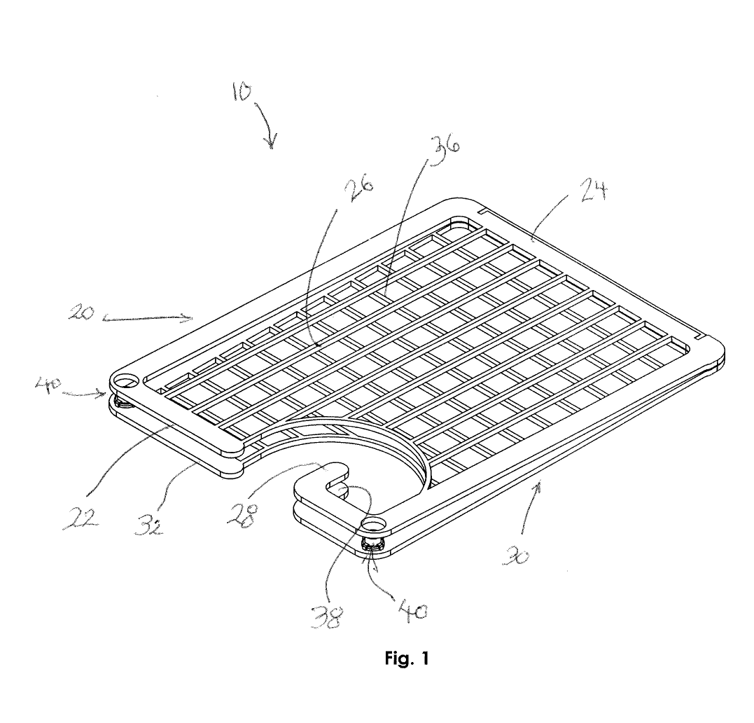

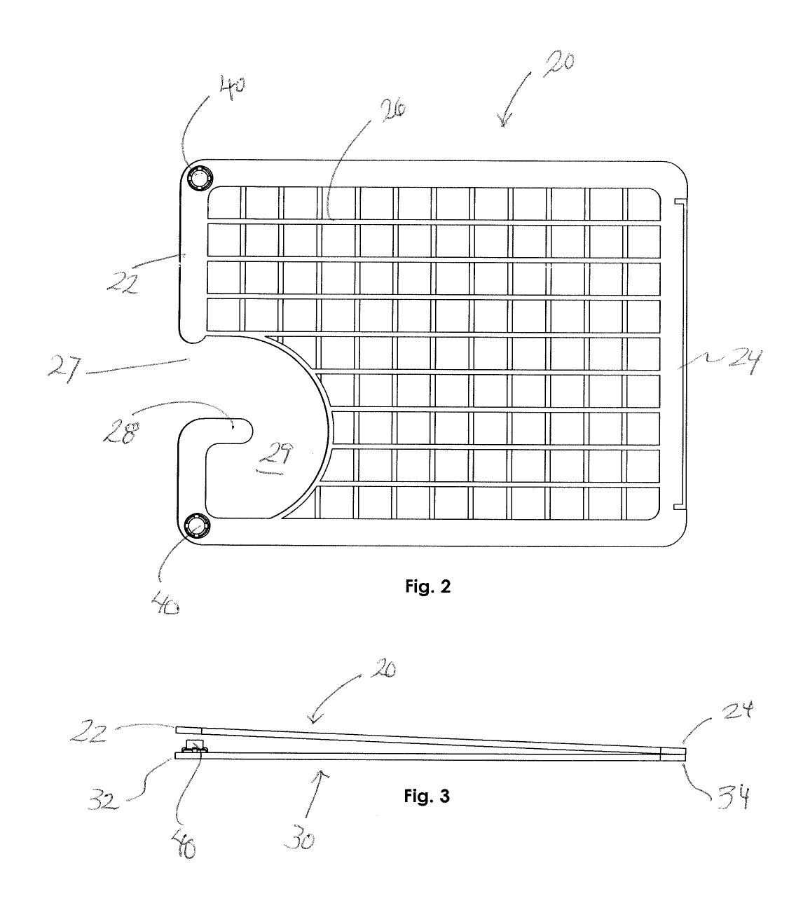

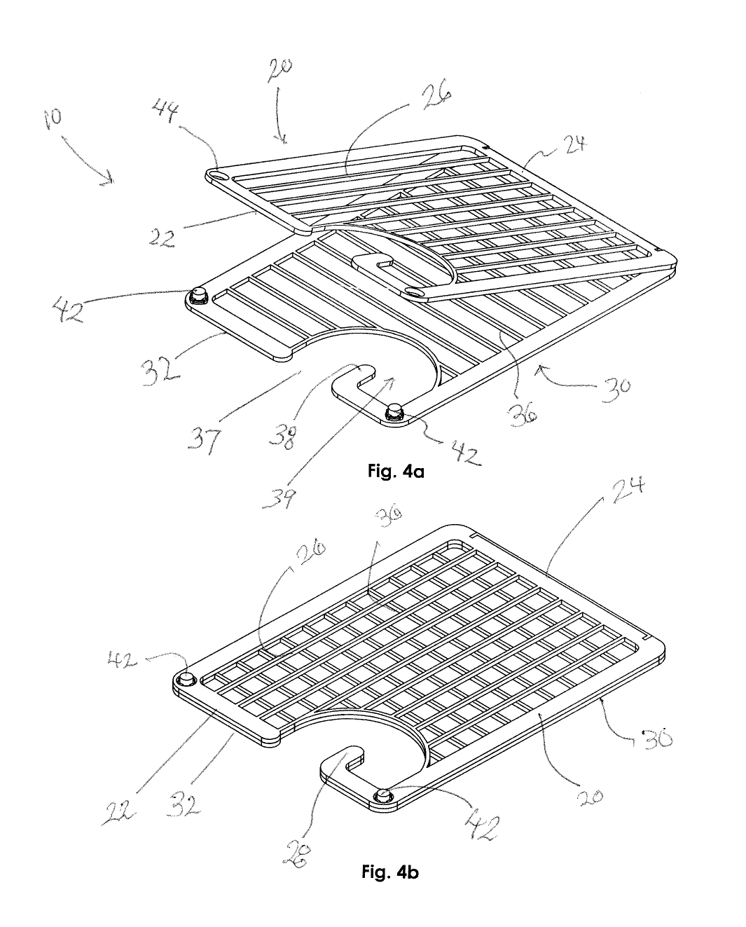

An odor mitigating caddy according to a preferred embodiment of the present invention will now be described in detail with reference to FIGS. 1 to 4c of the accompanying drawings. The odor mitigating caddy 10 includes a first frame member 20 (“upper frame structure), a second frame member (“lower frame structure”), and respective first and second hook members 28, 38.

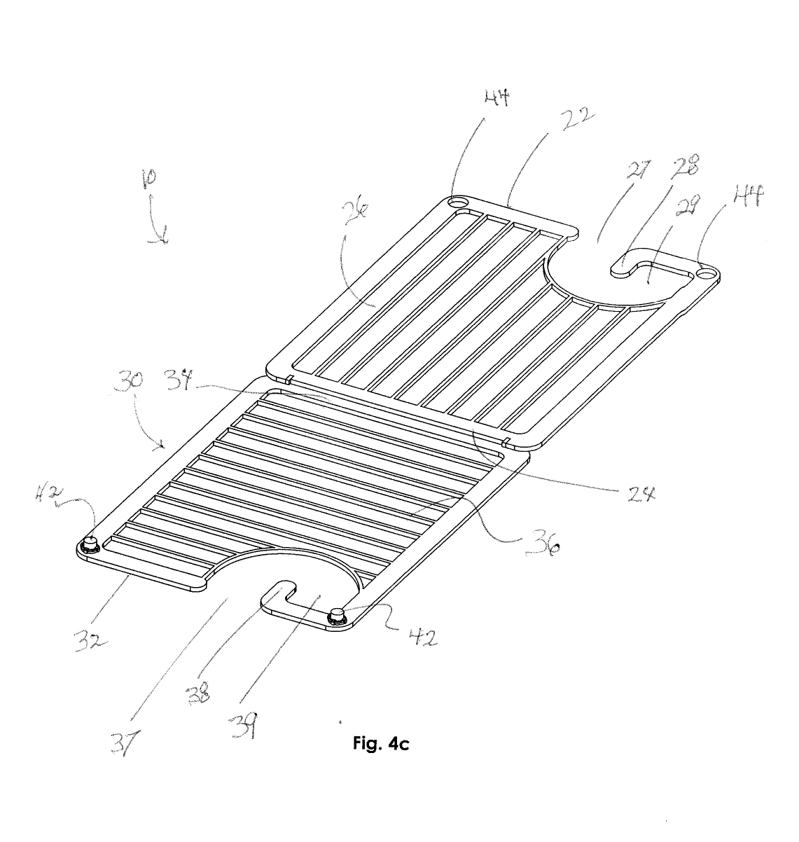

The first frame member 20 includes opposed front 22 and rear 24 edges. In like manner, the second frame member 30 includes opposed front 32 and rear 34 edges. Respective rear edges 24, 34 are pivotally coupled together such that the frame members are selectively movable between a closed configuration at which respective front edges 22, 32 are adjacent and immediately proximate one another (FIG. 4b) and an open configuration in which respective front edges 22, 32 are displaced and separated from one another (FIG. 4a). It is understood that an odor absorbing sheet, fragrance sheet, or other porous odor mitigating medium may be inserted between the frame members when the respective frame members are at the open configuration. The odor mitigating medium 12 is held in a substantially stationary position between the frame members 20, 30 when they are again positioned at the closed configuration. The rear edges 24, 34 may have a singular construction in the form of a living hinge, although separate construction connected by a traditional hinge would also work.

The configuration of the odor mitigating caddy 10 is defined by respective peripheral edges of respective frame members. In the preferred embodiment, the frame members have a rectangular or square configuration although a circular, oval, triangular or irregular shape configuration would also work. In any case, the frame members are substantially symmetrical in shape and design. More particularly, the first frame member 20 includes first peripheral edges and the second frame member 30 includes second peripheral edges generally aligned with the first peripheral edges so that the odor mitigating caddy 10 has a uniform and symmetrical configuration. Each frame member includes a porous grate structure extending between respective peripheral edges so that ambient air is able to move freely and efficiently into, out of, and through the frame members. Specifically, the first frame member 20 includes a first grate member 26 extending substantially between the first peripheral edges of the first frame member 20. The second frame member 30 includes a second grate member 36 extending substantially between the second peripheral edges of the second frame member 30. Each grate member may be a wire frame having wires or support members in a single direction such that, together, they form a horizontal-vertical pattern or, alternatively, each grate member may include both horizontal and vertical support members arranged in a grid-like pattern. In any case, the grate members cooperate to minimize any migration of a sheet of odor mitigating or fragrance media.

In another aspect, a first hook member 28 may be coupled to a respective peripheral edge of the first frame member 20 and is configured to hang the odor mitigating caddy 10 from a building support structure, e.g. a closet rail, corresponding hook or fastener, appliance handle, or other structure where odor mitigation is desired. More particularly, a respective peripheral edge of the first frame member 20 may include a bent portion extending inwardly into a first interior area 29 defined by the first grate member 26. In the preferred embodiment, the first interior area 29 and first hook member 28 (which is formed by the bent portion) are adjacent the front edge 22 of the first frame member 20. The front edge 22 of the first frame member 20 defines a first outlet opening 27 which enables access into the first interior area 29. It is understood that the first interior area 29 and first outlet opening 27 are essentially voids that enables the first hook member 28 to be hooked onto an external structure.

Similarly, a second hook member 38 may be coupled to a respective peripheral edge of the second frame member 30 and is configured to hang the odor mitigating caddy 10 from a building support structure, e.g. a closet rail, corresponding hook or fastener, appliance handle, or other structure where odor mitigation is desired. More particularly, a respective peripheral edge of the second frame member 30 may include a bent portion extending inwardly into a second interior area 39 defined by the first grate member 26. In the preferred embodiment, the second interior area 39 and second hook member 38 (which is formed by the bent portion) are adjacent the front edge 32 of the second frame member 30. The front edge 32 of the second frame member 30 defines a second outlet opening 37 which enables access into the first interior area 29. It is understood that the second interior area 39 and second outlet opening 37 are essentially voids that enables the second hook member 38 to be hooked onto an external structure. The second hook member 38, second interior area 39, and second outlet opening 37 are aligned with corresponding structures of the first frame member 20 described above.

In another aspect, at least one fastener combination is coupled to corresponding respective edges of the first and second frame members 20, 30. Preferably, one fastener combination is mounted to one end of respective front edges of respective frame members while a second fastener combination is mounted to a second (opposed) end of respective front edges. Each fastener combination may include a first element 42 coupled to one front edge and a complementary receiver element 44 coupled to a corresponding front edge. For example, suitable fastener combinations include snaps, hook-and-loop fasteners, magnets, clasps, hooks, friction-fit structures, and the like. In use, the fastener combinations securely maintain the frame members in the closed configuration until overcome manually to move to the open configuration.

In use, the first and second frame members 20, 30 may be pivotally moved to the open configuration and a dryer sheet (or other fragrance or odor mitigating medium) inserted. Then, the frame members may be moved back to the closed configuration to prevent migration of the sheet. The frame members may be locked in place as described above. The odor mitigation caddy 10 may then be hung from a closet rail, placed in an attic, automobile trunk, or even tossed into a clothes dryer so as to deodorize or impart a fragrance to a surrounding area as desired.

It is understood that while certain forms of this invention have been illustrated and described, it is not limited thereto except insofar as such limitations are included in the following claims and allowable functional equivalents thereof.

1. An odor mitigating caddy for securing and protecting porous media, comprising:
a first frame member having opposed front and rear edges;
a second frame member having opposed front and rear edges, said rear edges being pivotally coupled together such that said first and said second frame members are movable between a closed configuration at which said front edges, respectively, are generally adjacent one another and an open configuration at which said front edges, respectively, are separated from one another;
wherein said first frame member is defined by first peripheral edges and includes a first grate member extending between said first peripheral edges thereof;
wherein said second frame member is defined by second peripheral edges and includes a second grate member extending between said second peripheral edges thereof;
a first hook member coupled to a respective peripheral edge of said first frame member.

2. The odor mitigating caddy as in claim 1, further comprising a second hook member coupled to a respective peripheral edge of said second frame member, said second hook member being aligned with said first hook member.

3. The odor mitigating caddy as in claim 1, wherein said respective peripheral edge of said first frame member includes a bent portion extending into a first interior area defined by said first grate member, said bent portion forming said first hook member.

4. The odor mitigating caddy as in claim 2, wherein:
said respective peripheral edge of said first frame member includes a bent portion extending into a first interior area defined by said first grate member, said bent portion forming said first hook member;
said respective peripheral edge of said second frame member includes a bent portion extending into a second interior area defined by said second grate member, said bent portion forming said second hook member.

5. The odor mitigating caddy as in claim 1, wherein the porous media is held between said first frame member and said second frame member when said first and second frame members are at said closed configuration.

6. The odor mitigating caddy as in claim 4, wherein:
said bent portion of said respective peripheral edge of said first frame member defines a first outlet opening allowing access to said first interior area;
said bent portion of said respective peripheral edge of said second frame member defines a second outlet opening allowing access to said second interior area.

7. The odor mitigating caddy as in claim 4, wherein:
said respective peripheral edge of said first frame member is said front edge of said first frame member;
said respective peripheral edge of said second frame member is said front edge of said second frame member.

8. The odor mitigating caddy as in claim 4, further comprising at least one fastener combination coupled to said respective peripheral edge of said first and second frame members, respectively, said at least one fastener combination selectively holding said first and second frame members at said closed configuration or allowing said first and second frame members to move to said open configuration.

9. The odor mitigating caddy as in claim 1, wherein said first frame member is pivotally coupled to said second frame member with a living hinge.

10. The odor mitigating caddy as in claim 4, wherein said first grate member and said second grate members are porous.

11. An odor mitigating caddy for securing and protecting porous media, comprising:
a first frame member having opposed front and rear edges;
a second frame member having opposed front and rear edges, said rear edges being pivotally coupled together such that said first and said second frame members are movable between a closed configuration at which said front edges, respectively, are generally adjacent one another and an open configuration at which said front edges, respectively, are separated from one another;
wherein said first frame member is defined by first peripheral edges and includes a first grate member extending between said first peripheral edges thereof;
wherein said second frame member is defined by second peripheral edges and includes a second grate member extending between said second peripheral edges thereof;
a first hook member coupled to a respective peripheral edge of said first frame member; and
a second hook member coupled to a respective peripheral edge of said second frame member, said second hook member being aligned with said first hook member.

12. The odor mitigating caddy as in claim 11, wherein:
said first grate member defines a first interior area adjacent said front edge of said first frame member;
said second grate member defines a second interior area adjacent said front edge of said second frame member;
said respective peripheral edge of said first frame member includes a bent portion extending into said first interior area defined by said first grate member, said bent portion forming said first hook member;
said respective peripheral edge of said second frame member includes a bent portion extending into said second interior area defined by said second grate member, said bent portion forming said second hook member.

13. The odor mitigating caddy as in claim 12, wherein:
said bent portion of said respective peripheral edge of said first frame member defines a first outlet opening allowing access to said first interior area;
said bent portion of said respective peripheral edge of said second frame member defines a second outlet opening allowing access to said second interior area;
said first outlet opening being aligned with said second outlet opening.

14. The odor mitigating caddy as in claim 11, wherein:
said respective peripheral edge of said first frame member is said front edge of said first frame member;
said respective peripheral edge of said second frame member is said front edge of said second frame member.

15. The odor mitigating caddy as in claim 11, further comprising at least one fastener combination coupled to said respective peripheral edge of said first and second frame members, respectively, said at least one fastener combination selectively holding said first and second frame members at said closed configuration or allowing said first and second frame members to move to said open configuration.

16. The odor mitigating caddy as in claim 11, wherein said first frame member is pivotally coupled to said second frame member with a living hinge.

17. The odor mitigating caddy as in claim 11, wherein said first grate member and said second grate members define a plurality of apertures for efficient air transfer.
